(19) United States
(12) Patent Application Publication
(10) Pub. No.: US 2006/0122102 A1
(43) Pub. Date: Jun. 8, 2006

(54) Methods and preparations for curing critically ill patients

(75) Inventors: Greta Van Der Berghe, Grez-Doiceau (BE); Steffen Thiel, Risskov (DK); Troels Krarup Hansen, Arhus N (DK)

Correspondence Address: BROWDY AND NEIMARK, P.L.L.C.;624 NINTH STREET, NW, SUITE 300, WASHINGTON, DC, 20001-5303, US

(73) Assignees: K.U. Leuven Research and Development, Leuven (BE); Aarhus University Hospital, Hojbjerg (DK)

(21) Appl. No.: 10/528,802

(22) PCT Filed: Sep. 23, 2003
(86) PCT No.: PCT/BE03/00158

§ 371 Date: Mar. 23, 2005

(30) Foreign Application Priority Data
Sep. 23, 2002 (GB) 0222014.3

(51) Int. Cl. A61K 38/16

(52) U.S. Cl. 514/8

(57) ABSTRACT

The present invention pertains to the use of a blood mannan-binding lectin (MBL) regulator for the manufacture of a life saving drug to treat or cure a critically ill patient. It further involves the use of measurements of MBL to predict mortality in critically ill ICU patients. One further aspect of present invention is to the use of monomers and oligomers of MBL in prophylactic and/or curative treatment of patients admitted to intensive care units (ICUs).

### TECHNICAL FIELD

The present invention pertains to the use of a blood mannan-binding lectin (MBL) regulator for the manufacture of a life saving drug to treat or cure a critically ill patient. It further claims the use of measurements of MBL to predict mortality in critically ill ICU patients. One further aspect of present invention is to the use of monomers and oligomers of MBL in prophylactic and/or curative treatment of patients admitted to intensive care units (ICUs).

### BACKGROUND OF THE INVENTION

Mortality among patients with prolonged critical illness exceeds 20 percent, with most deaths being attributable to sepsis and multiple-organ failure (Van den Berghe G et al., N Engl J Med 2001;345(19):1359-67; Takala J, et al. N Engl J Med 1999;341(11):785-92). Each year, these conditions affect more than 500 000 patients in the United States alone (Wheeler A P et al. N. Engl. J. Med. 1999;340(3):207-14). An increased susceptibility to severe infections during critical illness, as well as adverse effects of an excessive systemic inflammatory response on organ function may be operative.

The average serum concentration of MBL in the adult population is between 1000-2000 ng/ml with very large variations (Turner M W and Hamvas R M. Rev. Immuno-genet. 2000;2(3):305-22). The between-subjects differences in serum concentrations are primarily caused by genetic factors. Point mutations within exon 1 as well as in the promoter region of the MBL gene occur with high incidence. As a consequence, approximately one third of the population have MBL concentrations below 500 ng/ml, and more than 10% have concentrations below 50 ng/ml (Steffensen R, et al. J. Immunol. Methods 2000;241(1-2):33-42). Normally, within-subject variations of MBL levels are very small (Hansen T K, et al J. Clin. Endocrinol. Metab 2001;86(l1):5383-8), but serum concentrations increase during acute phase responses (Thiel S, et al. Clin. Exp. Immunol. 1992;90(1):31-5) and can be specifically induced by growth hormone (GH) administration (Hansen T K, et al. J. Clin. Endocrinol. Metab 2001;86(11):5383-8). Deficiency of MBL is associated with an increased incidence of infections (Super M, et al. Lancet 1989;2(8674):1236-9; Koch A, et al. JAMA 2001;285(10):1316-21; Summerfield J A, et al. BMJ 1997;314(7089):1229-32; Garred P, et al Lancet 1995;346(8980):941-3), but due to the redundancy of the immune system the increased risk may only be apparent if other coexisting immunological abnormalities are present. In line with this, it was recently reported that low levels of MBL in patients receiving cancer chemotherapy are associated with an increased frequency of febrile neutropenic episodes and severe infections (Neth O, Hamn I, et al. Lancet 2001;358(9282):614-8.; Peterslund N A, et al. Lancet 2001;358(9282):637-8). The impact of MBL concentrations on the course of disease in otherwise immunocompetent critically ill patients has not been studied yet and is an object present invention.

By present study was investigated how MBL affects outcome in critically ill patients. In all patients, irrespective of the treatment, the MBL concentrations increased significantly with the time of intensive care (P<0.0001), a rise that was independent of the baseline MBL concentration, but attributable to the survivors. By present invention it was demonstrated that low MBL levels were at negative outcome in critically ill patients. MBL concentrations at baseline were almost three times higher in survivors than in non-survivors (p=0.04).

This invention demonstrates that restoring MBL levels in critically ill patients to normal levels, preferable to a level above 250 μg/l serum, more preferably to a level above 500 μg/l serum, more preferably to a level above 1000 ng/ml and most preferably between 1000 ng/ml and 2000 ng/ml can be used to improve outcome such as the survival of critically ill patients in the intensive care unit (ICU). However, after this invention, it will be clear for the man skilled in the art that also active MBL derivatives or compounds of the group of biologically active substances, which stimulate late hepatocytes to synthesise and/or release of MBL and consequently increase the level of circulating MBL can be used to obtain the same outcome.

The MBL is obtainable from various sources.

Mannan-binding lectin was first isolated from human serum in 1983 (Kawasaki N. et al, J. Biochem (Tokyo), 1983, 94, 937-947) by affinity chromatography on mannan-Sephrarose (mannan coupled to a Sepharose matrix) in the presence of Ca-ions. Elution of MBL from the affinity column was performed by means of EDTA Improved methods of manrose binding protein purification have been described in Ezewkowitz Raymond U.S. Pat. No. 5,270,199; S. M. Tan et al Biochemical journal Vol. 319, no 2, 15 October 1996, pages 329-332; R. Koppel et al. Journal of chromatography B.: Bio-medical Applications., vol. 662, no 2, 1994 pages 191-196.; D. C. Kilpatrick Transfusion Medicine, vol. 7 , no. 4, 1997 pages 289-294 and WO 99/64453 Laursen Inga. MBL can be isolated by passage down a mannose-Sepharose column as described by Wild et al., Biochem. J., 210: 167-174 1983 or Drickamer et al., J. of Bio. Chemistry, 261: 6878-6887 1986. MBL can be produced in engineered cells. Recombinant MBL has been produced by mammalian cell culture (Ezekowitz, U.S. Pat. No. 5,270,1999) such as in myeloma cells, Chinese hamster ovary (CHO) cells, human hepatocytes, and human embryonic kidney (HEK) cells (Vorup-Jensen,-T et al. Int-Immunopharmacol. April2001; 1(4): 677-87) or by expression of MBL in methylotrophic yeast strains as for instance described in U.S. Pat. No. 6,337,193.

Clinical grade MBL has been obtained and shown to be safe upon infusion It has for instance already been demonstrated in patients with long disease that pooled human donor plasma derived MBL can safely be administered to patients (Garred,-Peter; et al Pediatr-Pulmonol. March 2002; 33(3): 201-7). Moreover, present invention demonstrated for the first time an efficient MBL therapy for critically ill patients in the ICU, which were not previously been immunocompromised (e.g. not immunosupressed after organ transplantation or by disease).

Furthermore it will be clear for the man skilled in the art that compounds of the group of biologically active substances which stimulate hepatocytes to synthesise and or release MBL and consequently increase the level of circulating MBL can be used to in a prophylactic or therapeutic treatment to improve outcome and survivability of critically ill patients in the ICU. Such compound with an activity of promoting the secretion of MBL were already well disclosed before the moment of this invention such as growth hormone (Hansen,-T-Ket al J-Clin-Endocrinol-Metab. November 2001; 86(11): 5383-8).

The general structure of MBL is shown in EP0375736B1 May 13, 1998 Aug. 5, 1988.

Nucleic acid, for example, DNA, encoding MBL can be isolated by standard techniques as for instance described in U.S. Pat. No. 5,270,199. For example, oligonucleotide probes specific for the nucleic acid may be constructed and used to probe either genomic or cDNA libraries, as described by Drickamer et al, J. Biol. Chem., 261:6878 (1986), Alternatively, gene fragments from related genes can be used as probes. Preferably, the probe is homologous to a region of the carbohydrate binding domain of MBL. The clones isolated by this technique contain engineered nucleic acid. Once isolated, the gene encoding MBL is useful for producing recombinant MBL, or peptide fragments thereof In addition, the nucleic acid can be modified by standard techniques in order to express modified peptides. For instance, a human liver cDNA library has been constructed by standard technique as described by Woods et al. Proc. Natl. Acad. Sci. USA. 5661, 1982. This library was probed using a gel purified radiolabelled rat MBL-C cDNA sequence digested with XhoI and EcoRI as described by Drickamer (J. Biol. Chem. 263:9557, 1988). Such probe can be used under non-stringent conditions to identify Potentially useful clones ( Kwiatkowski et al. 323 Nature 455, 1986; Messing et al. Proc. Nat. Acad. Sci. USA 74:3642, 1977). A MBL cDNA clone has for instance been used as a probe for human genomic library. Such library can for instance be constructed by standard techniques and clones, which hybridised under stringent conditions can be isolated.

Expression of MBL peptide fragments can be done by standard procedures. For example, the desired region of the MBL encoding DNA, preferably the cDNA, can be isolated from one of the above-described clones and inserted into any one of several standard expression vectors. A preferred region for expression is that encoding the carbohydrate binding lectin isolated from the coelornic fluid of a sea urchin A. crassispina (Giga et al., J. Biol. Chem 13: 6197, 1987); a chicken cartilage core proteoglycan protein (Shigaku et al., Proc. Natl. Acad. Sci. USA 83:5081, 1986) and the IgE Fc receptor (Ikuta et al., Proc. Natl. Acad. Sci. USA 84:819, 1987).

Antibodies to such expressed peptides or to MBL itself can be produced by standard techniques. They may be monoclonal or polyclonal and are useful for identification of the peptides within animal serum or in clinical diagnostic tests.

A number of carbohydrate-binding proteins (lectins), are known in man. One group is the C-type lectins. The C-type lectins contain a calcium-dependent carbohydrate recognition domain (a C-type CRD) (Weis W I, Taylor M E and Drickamer K (1998) Immunological Reviews 163: 19-34). Mannan-binding lectin (MBL), synonymous to mannose-binding lectin, mannan-binding protein or mannose-binding protein (MBP), belongs to the subgroup of C-type lectins, termed collecting. These soluble proteins are composed of subunits presenting three CRDs attached to a collagenous stalk (Holmskov, U., Malhotra, R., Sim, R. B., and Jensenius, J. C. (1994) Immunol. Today 15:67-74). MBL interact with carbohydrates presented by a wide range of micro-organisms and accumulating evidence shows that it plays an important role in the innate immune defence (Turner, M. W. (1996) Immunol. Today 17:532-540).

When bound to carbohydrate MBL is able to activate the complement system. The complement system may be activated via three different pathways: the classical pathway, the alternative pathway, and the mannan-binding lectin (MBL) pathway that is initiated by the binding of MBL to carbohydrates presented by micro-organisms. The components of the alternative pathway and of the MBL pathway are parts of the innate immune defence, also termed the natural or the non-clonal, immune defence, while the classical pathway involves cooperation with antibodies of the specific immune defence (Janeway C A, et al (1999) Immunobiology, the immune system in health and disease, Fourth Edition, Churchill Livingstone). MBL is synthesised in the liver by hepatocytes and secreted into the blooi It binds to carbohydrate structures on bacteria, yeast, parasitic protozoa and viruses, and exhibits antibacterial activity through killing of the micro-organisms by activation of the terminal, lytic complement components or through promotion of phagocytosis (opsonization). The sertiform structure of MBL is somewhat similar to the bouquet-like structure of C1q, the immunoglobulin-binding subcomponent of the first component in the classical pathway (Turner, M. W. (1996) Immunol. Today 17:532-540). C1q is associated with two serine proteases, C1r and C1s, to form the C1 complex. Simlarly, MBL is associated with the serine proteases MASP-1 (Matsushita, M. and Fujita, T (1992). J. Exp. Med. 176:1497-1502), MASP-2 (Thiel S, et al. Nature, 386(6624): 506-510), MASP-3 (Dahl MR, et al. Immunity. 2001;15(1):127-35) and an additional protein called MAp19 (Stover C M, et al J Immunol 162: 3481-3490). MASP-1, MASP-2 and MASP-3 have modular structures identical to those of C1r and C1s (Dahl M R, et al Immunity. 2001;15(l):127-35). The binding of MBL to carbohydrates induces the activation of MASP-1, MASP-2 and MASP-3. Activated MASP-2 then generates the C3 convertase, C4bC2b, through cleavage of the complement factors C4 and C2 (Thiel Set al (1997) Nature, 386(6624): 506-510). Reports suggest that MASP-1 may activate C3 directly (Matsushita M. et al J. Exp. Med. 1992; 176 (6): 1497-502). The possible substrates for MASP-3 is unknown. Nothing is known about the stoichiometry and activation sequence of the MBL/MASP complexes. ML has also been characterised in other animals such as rodents, cattle, chicken and monkeys.

The human MBL protein is composed of up to 18 identical 32 kDa polypeptide chains (Lu, J., et al. (1990) J. Immunol. 144:2287-2294), each comprising a short N-terminal segment of 21 amino acids including three cysteine residues, followed by 7 repeats of the collagenous motif Gly-X—Y interrupted by a Gln residues-followed by another 12 Gly-X—Y repeats. A small 34 residue ‘neck-region’ joins the C-terminal Ca²⁺-dependent lectin domain of 93 amino acids with the collagenous part of the molecule (Sastry, K., et al. (1989) J. Exp. Med. 170:1175-1189).

The collagenous regions of the polypeptide chain combines in subunits composed of these polypeptide chains which are covalently linked by disulphide bridges, both between individual subunits and between the polypeptide chains in each subunit (Turner, M. W. (1996) Immunol. Today 17:532-540).

The position of these disulphide bridges has, however, not been fully resolved. SDS-PAGE analysis under non-reducing conditions of MBL shows bands with an apparent molecular weight (m.w.) larger than 200 kDa presumably representing blocks of 3×3, 4×3, 5×3 and even 6×3 complexes of covalently assembled subunits (Lu, J., et al. (1990) J. Immunol. 144:2287-2294).

The actual number of subunits in the natural human MBL protein has been controversial. Lipscombe et al. obtained data by use of ultracentrifugation suggesting 25% of human serum MBL to be made of 2-3 trimers and only a minor fraction reaching the size of 6 trimers (Lipscombe, R. J., et al. (1995) Immunology 85:660-667). The relative quantification was carried out by densitometry of Western blots developed by chemiluminescence. By SDS-PAGE analysis of fractions from ion exchange chromatography it was found that the predominant species of covalently linked MBL subunit chains consisted of tetramers while only pentameric or hexameric complexes activated complement (Lu, J., et al. (1990) J. Immunol. 144:2287-2294.). Gel permeation chromatography (GPC) analysis, in contrast, suggests that MBL is comparable in size with the C1 complex GPC can be carried out under conditions which allow for a study of the importance of weak protein-protein interactions in the formation of MBL molecules and, in combination with standard MBL assay techniques, also allows for unbiased determination of the MBL content in the GPC fractions.

The concentration of MBL in human serum is largely genetically determined, but reportedly increases up to threefold during acute phase reactions (Thiel S. et al. (1992) Clin Exp Immunol 90: 31-35). Three mutations causing structural alterations as well as two mutations in the promotor region are associated with MBL deficiency (Madsen, H. O., et al. (1994) Immunogenetics 40:37-44). MBL deficiency is associated with susceptibility to a variety of infections. Examination of five adult individuals with unusual and severe infections showed three to be homozygous for structural MBL mutations and two to be heterozygous (Summerfield J A, et al (1995) Lancet 345: 886-889). Investigation of 229 children referred to the Danish National Hospital because of non-HIV-related immunodeficiency showed a tenfold higher frequency of homozygosity for structural MBL mutant alleles than seen in a control group (Garred P, et al. (1995) Lancet 346: 941-943). Allotyping of 617 consecutively hospitalized children at St Mary's Hospital in London showed significantly higher frequency of homozygosity and heterozygosity for mutant allotypes in the infected children than in the noninfected (Summerfield J A, (1997) BioMed J 314: 1229-1232).

MBL can bind to a wide range of oligosaccharides. As MBL does not usually recognise self-determinants, but is well suited to interactions with microbial cell surfaces presenting repetitive carbohydrate determinants. In vitro, yeast (Canidida albicans and Cryptococcus neoformans), viruses ( HIV-1, HIV-2, HSV-2, and various types of influenza A) and a number of bacteria have been shown to be recognised by MBL. In the case of some bacteria, the binding with MBL is impaired by the presence of a capsule (van Emmerik, L C, et al. (1994) Clin. Exp. Immunol. 97:411-416). However, even encapsulated bacteria (Neisseria meningitidis) can show strong binding of MBL (Jack D L, et al. (1998) B. J Immunol 160: 1346-1353),

The micro-organisms, which infect MBL deficient individuals, represent many different species of bacterial, viral and fungal origin (Turner, M. W. (1996) Immunol. Today 17:532-540; Summerfield J A, et al. (1997) BioMed J 314: 1229-1232). MBL may be a general defence molecule against most bacteria, and thus be a reason why so many bacteria are non-pathogenic.

While accumulating data support the notion of a protective effect of MBL there are also observations suggesting that infections with some micro-organisms, notably intracellular pathogens, attain a higher frequency in MBL sufficient than in MBL deficient individuals (Garred, P, et al. (1994) Eur. J. Immunogen. 21:125-131 and Hoal-Van Helden E G, et al (1999) Pediatr Res 45:459-64; Hoal-Van Helden E G, et al. (1999) Pediatr Res 45:459-64). This is in concordance with the results of an animal experiment, where an increased number of HSV-2 were found in the liver of mice pre injected with human MBL (Fischer, P B, et al. (1994) Scand J Immunol 39:439-445).

We investigated whether a low MBL level affects outcome in non-immunocompromised critically ill patients. Protracted critical illness is associated with substantial metabolic derangement and a high risk of death (Van den Berghe G. et al 2000; 143 (1): 1-13). Mortality among patients with prolonged critical illness exceeds 20 percent (Van den Berghe G, et al. N Engl J Med 2001;345(19):1359-67; Takala J, et al. N Engl J Med 1999;341(11):785-92), with most deaths being attributable to sepsis and multiple-organ failure. Each year, these conditions affect more than 500,000 patients in the United States alone (Wheeler A P and Bernard G R. N. Engl. J. Med. 1999;340(3):207-14). An increased susceptibility to severe infections during critical illness, as well as adverse effects of an excessive systemic inflammatory response on organ function may be operative.

Previously MBL infusion has been suggested for treating MBL deficiency in immuno-compromised individuals. Immunocompromised is used in its normal meaning, i.e. an individual not being capable of evoking an immune response towards an infection MBL deficiency has often been defined by an arbitrary level of about 50 ng/ml This level is often identical with the sensitivity of various MBL test assays, and the level has therefore been set as the level for which substantially no MBL could be detected In the case of patients treated with chemotherapy a level of 500 ng/ml has been suggested as defining MBL deficiency in this condition.

By the present invention it has been demonstrated that administrating MBL to ICU admitted patients may reduce the risk of death of subjects that were non-immunocompromised subjects during prolonged ICU stay. The patients in question may have a MBL level below 500 ng/ml serum. Clinical grade MBL is available and has been shown to be safe upon infusion (Garred,-Peter; et al. Pediatr-Pulmonol. March 2002; 33(3): 201-7). Production of recombinant MBL conceivably having a structure and an activity similar to that of native MBL has been attained (patent application PA 1999 00668/C5/KH).

### SUMMARY OF THE INVENTION

The invention features the use of MBL, purified from natural sources or from material produced by recombinant technologies, or by any other suitable MBL-producing cell line, for the treatment of individuals admitted to ICUs. The MBL may be given before or after start of the treatment and for any duration of time deemed suitable.

The invention in one aspect relates to treatment of individuals admitted to ICUs, critically ill patients or to treatment of individuals who are at risk of prolonged ICU admission due to procedures/treatment known to be associated with allocations to ICUs (e.g. major surgery).

Accordingly, complications arising during ICU-stay are likely to expose the individual in question to a higher risk of inflammatory conditions and indeed death. It is possible according to the invention to prophylactically treat the patients before or during procedures/treatments (e.g. major surgery) known to be associated with a risk of prolonged ICU admission. By prophylactically treating the ICU-complications before or during a treatment known to be associated with a risk of prolonged ICU admission it is possible to reduce the mortality from sepsis and septic shock arising during the ICU-stay.

In another aspect the present invention is related to the use of a composition comprising at least one mannan-binding lectin (MBL) polypeptide monomer, or at least one oligomer comprising at least one mannan-binding lectin (MBL) polypeptide monomer, in the manufacture of a medicament for prophylactic, ameliorating or curative treatment of a condition obtained during intensive care, in an individual initially having plasma levels of MBL below 500 ng/ml.

In yet another aspect there is provided a method to predict the risk of fatal outcome in an individual allocated to ICUs through measurements of the concentration of MBL in plasma or serum obtained from the individual and estimation of the probability on the basis of the measured concentration.

### DEFINITIONS

The term “systemic inflamatory response syndrome (SIRS)”, as used herein refers to the uncontrolled disease process which ensues an initial insult and which gives rise to a multi system disturbance secondary to inflammatory mediators released during shock. It can mean a response to an inflammation or injury that can be infections or non-infectious, defined by having two of the following: 1) Temperature above 38 degrees C. or less than 36 degrees C., 2) Heat rate >90, 3) Respiration rate >20 or Paco2<32 torr, 4) WBC>12,000/mm3 or <40000, or >10% bands.

The term “Sepsis”, as used herein refers to “SIRS”, as described above, which is particularly caused by an infectious insult leading to the initial shock phase.

The term “Bacteraemia”, as used herein means the presence of bacteria in the blood-stream detected by blood cultures.

The term “Septic shock” as used herein means Sepsis with a systolic BP <90 mm or drop of 40 mm Hg from baseline value in absence of other causes. Severe sepsis may cause organ dysfunction.

The term “Critical ill patients” (CIP) as used herein means patients which have sustained or are at risk of sustaining acutely life-threatening single or multiple organ system failure due to disease or injury, a patient who is being operated and where complications supervene, and a patient who has been operated in a vital organ within the last week subject to major surgery within the last week. Usually and preferably, these conditions necessitate prolonged minute to minute therapy an/or observation, usually and preferably in an intensive care unit (ICU) which is a part of an hospital or alike capable of providing a high level of intensive therapy in terms of quality and immediacy. Critical illness might be explained as a disease or state in patients in which death is possible or imminent and whereby the patients are maintained under total or partial parenteral nutrition in an intensive care unit (ICU), a hospital facility or alike for provision of intensive nursing and medical care, characterised by high quality and quantity of continuous monitoring. In a more restricted sense, the term a “critically ill patient”, as used herein refers to a patient who has sustained or are at risk of sustaining acutely life-threatening single or multiple organ failure due to disease or injury, or a patient who is being operated and where complications supervene. In a even more restricted sense, the term a “critically ill patient”, as used herein refers to a patient who has a sustained or are at risk of sustaining acutely life-threatening single or multiple organ system failure due to disease or injury. Similarly, this definition applies to similar expressions such as ‘critical illness in a patient’ and a ‘patient is critical ill’. Examples of a critically ill patient is a patient in need of cardiac surgery, cerebral surgery, thoracic surgery, abdominal surgery, vascular surgery, or transplantation, or a patient suffering from neurological diseases, cerebral trauma, respiratory insufficiency, abdominal peritonitis, multiple trauma, severe burns, or critical illness polyneuropathy.

### DESCRIPTION OF THE ILLUSTRATIVE EMBODIMENT

Indication

Previously MBL infusion has been suggested for treating MBL deficiency in imuno-compromised individuals. Immunocompromised is used in its normal meaning, i.e. an individual not being capable of evoking an immune response towards an infection. MBL deficiency has often been defined by an arbitrary level of about 50 ng/ml. This level is often identical with the sensitivity of various MBL test assays, and the level has therefore been set as the level for which substantially no MBL could be detected. In the case of patients treated with chemotherapy a level of 500 ng/ml has been suggested as defining MBL deficiency in this condition

By the present invention administrating MBL to ICU admitted patients may reduce the risk of death from sepsis and septic shock during prolonged ICU stay. The patients in question may have a MBL level below 500 ng/ml serum.

Also the treatment of ICU admitted individuals may be conducted by administering MBL to these individuals in combination with relevant antibiotics, anti-viral agents or anti-fungal agents.

In particular, individuals at risk of prolonged ICU admission due to a surgical or medical treatment will benefit from prophylactic treatment with MBL before, during and possibly also after the treatment in order to reduce the mortality from complications (sepsis, septic shock or multiple organ deficiency ) arising during the ICU-stay.

Generally all individuals being allocated to ICUs having a MBL level below 500 ng/ml should be treated with MBL to reduce the risk of death from complications (sepsis and septic shock) arising during the ICU-stay, independent of their specific MBL level. Consequently, in particular individuals having a MBL level below 400 ng/ml will benefit, such as individuals having a MBL level below 300 ng/ml, such as individuals having a MBL level below 200 ng/ml, such as individuals having a MBL level below 100 ng/ml, such as individuals having a MBL level below 50 ng/mL

Thus, the present invention in particular relates to the use of MBL polypeptides or blood MBL increasing factors for manufacturing of a medicament for the treatment of individuals having a MBL level in serum in the range of 0-500 ng/ml. The condition may be due to the treatments at ICUs. However, the condition may also be due to a surgical or medical treatment known to be associated with a risk of prolonged ICU admission.

The MBL deficiency may be without known genesis, either acquired or inherited. Individuals, having a MBL level below 500 ng/ml will benefit from MBL treatment in general in order to prevent ICU induced conditions.

According to one embodiment, the present invention relates to the use of MBL or blood MBL stimulators for the manufacture of a life saving drug to treat or cure a critically ill patient.

According to another embodiment, the present invention relates to a use of MBL or blood MBL stimulators for the manufacture of a medicament to treat or cure a critically ill patient.

According to yet another embodiment, the present invention relates to the use of MBL or blood MBL stimulators for the manufacture of a medicament to prevent that a patient becomes critical ill.

According to a further embodiment, the present invention relates to a use of MBL or blood MBL stimulators for the manufacture of a medicament to increase the survival rate of a critically ill patient.

According to a further embodiment, the present invention relates to a use of MBL or blood MBL stimulators for the manufacture of a medicament to reducing the time a critically ill patient ill patient stays within a hospital, for example within an ICU.

According to a further embodiment, the present invention relates to a use of MBL or blood MBL stimulators for the manufacture of a medicament to prevent treat or cure sepsis and/or its mediators, especially in a critically ill patient.

According to a further embodiment, the present invention relates to a use of MBL or blood MBL stimulators for the manufacture of a medicament to reduce mortality, hospitality stay, bacteraemia, need for ventilatory support, need for dialysis.

In a further embodiment of present invention, the critically ill patient is a patient in need of cardiac surgery.

In a further embodiment of present invention, the critically ill patient is a patient in need of cerebral surgery.

In a further embodiment of present invention, the critically ill patient is a patient in need of thoracic surgery.

In a further embodiment of present invention, the critically ill patient is a patient in need of abdominal surgery,

In a further embodiment of present invention, the critically ill patient is a patient in need of vascular surgery.

In a further embodiment of present invention, the critically ill patient is a patient in need of transplantation

In a further embodiment of present invention, the critically ill patient is a patient suffering from neurological diseases.

In a further embodiment of present invention, the critically ill patient is a patient suffering from cerebral trauma.

In a further embodiment of present invention, the critically ill patient is a patient suffering from respiratory insufficiency.

In a further embodiment of present invention, the critically ill patient is a patient suffering from abdominal peritonitis.

In a further embodiment of present invention, the critically ill patient is a patient suffering from multiple trauma.

In a further embodiment of present invention, the critically ill patient is a patient suffering from severe burns.

In a further embodiment of present invention, the critically ill patient is a patient suffering from critical illness neuropathy.

In a further embodiment of present invention, the critically ill patient is a patient being mechanically ventilated.

Furthermore, the present invention relates to a method of selling a substance or a composition of said substance, which stimulate the levels of circulating MBL in a subject by giving information about their novel utility, novel activity and/or novel pharmaceutical indications described herein. One method of selling such blood MBL regulator could be by telling a person, for example a physician, that MBL or a factor which stimulate hepatocytes to synthesise and/or release of MBL and consequently increase the level of circulating MBL maybe used to treat critically ill patients or to reduce the mortality from complications (sepsis and septic shock) arising during the ICU-stay. Alternatively, a method of selling a blood MBL regulators or MBL could be by distributing the above advertising and information, whereby the media are brochures, packaging material which is used for the customer package, any printed material/leaflet supplied with the drug, or patient information, labels, web sites, movies, advertising movies, videos, and the like.

Another method of selling a blood MBL regulators or MBL which is covered by the present claims is to support a speaker giving information about the novel utility, indication, and action of the blood glucose regulator according to the present invention or to support an author writing an article giving information about the novel utility, indication, and action of the a blood MBL regulators or MBL according to the present invention. Other variations hereof will be obvious for the skilled art worker, for example distributing and advertisement as the above.

MBL

The MBL composition used to manufacture a MBL medicament may be produced from any MBL source available. The MBL source may be natural MBL, whereby the MBL polypeptides are produced in a native host organism, meaning that MBL is produced by a cell normally expressing MBL. One usual method of producing a MBL composition is by extraction of MBL from human body liquids, such as serum or plasma.

In another aspect the MBL polypeptide oligomers are produced by a host organism not natively expressing a MBL polypeptide, such as by recombinant technology.

In a first embodiment the MBL source may be serum, from which an MBL composition is obtained by purifying serum, plasma, milk product, colostrum or the like by a suitable purification method, such as affinity chromatography using carbohydrate-derivatised matrices, such as mannose or mannan matrices. Such a method is discussed in WO99/64453 which is hereby incorporated by reference.

The MBL composition used to manufacture a MBL medicament preferably comprises MBL oligomers having a size distribution substantially identical to the size distribution of MBL in serum, such as a size distribution profile at least 80% identical to the size distribution profile of MBL in serum, more preferred a size distribution profile at least 90% identical to the size distribution profile of MBL in serum more preferred a size distribution profile at least 95% identical to the size distribution profile of MBL in serum.

The matrix may be derivatized with any carbohydrate or carbohydrate mixture whereto MBL binds. The matrix is preferably a mannose-, a fucose, a N-acetyl-glucosamin or a glucose derivatized matrix, such as most preferably a mannose matrix.

The selectivity of the carbohydrate-derivatized matrix is obtained by securing that the matrix as such, i.e the un-derivatized matrix has substantially no affinity to MBL polypeptides. This may be ensured when the matrix as such is carbohydrate-free.

The matrix may be in any form suitable for the chromatography, mostly in the form of beads, such as plastic beads.

After application of the MBL source the column is washed, preferably by using non-denaturing buffers, having a composition, pH and ionic strength resulting in elimination of or lowering of the amount of contaminating proteins, without eluting the MBL. Such as buffer may be TBS (10 mM Tris, 145 mM NaCl, pH 7.4) with calcium ions added. Elution of MBL is performed with a selective desorbing agent, capable of efficient elution of MBL, such as TBS, with added EDTA, and MBL oligomers are collected. Such a purification method is described in International patent application (WO0070043 and WO9937676).

In a preferred aspect a clinical grade MBL composition is obtained by using an MBL source produced by recombinant technology, wherein the MBL source is the culture media from culturing of MBL producing cells.

Thus, the present invention encompasses MBL produced by a process of producing a human recombinant mannan binding lectin (MBL) polypeptide, comprising the steps of:
preparing a gene expression construct comprising a DNA sequence encoding a human MBL polypeptide or a functional equivalent thereof
transforming a host cell culture with the construct,
cultivating the host cell culture, thereby obtaining expression and secretion of the polypeptide into the culture medium, followed by
obtaining a culture medium comprising human recombinant MBL polypeptides.

The culture medium comprising the human recombinant MBL polypeptides may then be purified as described above.

The gene expression construct may be produced by conventional methods known to the skilled person, such as described in U.S. Pat. No. 5,270,199.

In another embodiment the gene expression construct is prepared as described in Danish Patent application No: PA 1999 00668 or in International patent application (WO0070043) having the title “Recombinant Human Mannan Binding Lectin”).

The expression is preferably carried out in e.g. mammalian cells, the preparation according to the invention results from the use of an expression vector comprising intron sequence(s) form an MBL gene and at least one exon sequence. Regarding the transgenic animals as expression system this term is in this context animals which have been genetically modified to contain and express the human MBL gene or fragments or mimics hereof.

In addition to the purification method it is preferred that the gene expression construct and the host cell also favours production of higher oligomers, which has been found to be possible by using a gene expression construct comprising at least one intron sequence from the human MBL gene or a functional equivalent thereof.

In particular the MBL composition is used for treatment and/or prophylaxis of sepsis, septic shock or multiple organ failure which occurs in patients treated within the intensive care unit (ICA). Such patients may have post-surgical critical illness, post-traumatic critical illness or patients on ventilator in the ICU.

It is an object of present invention to use the MBL composition in a life saving treatment of critically ill patients.

A still further object of present invention is to use the MBL composition to reduce the time a critically ill patients stays within a ICU.

Another object of present invention is to use the MBL composition to suppress states of sepsis, septic shock or multiple organ failure.

Another object of present invention is to use the MBL composition to reduce the risk or likelihood from multiple organ failure with a proven septic focus on post-mortem examination in a critically ill patient.

Another object of present invention is to use the MBL composition to reduce the mortality, for example, in hospital mortality, in a critically ill patient.

Another object of the present invention is to use the MBL composition to reduce the likelihood of organ replacement therapy and/or organ failure (for instance renal) in a critically ill patient.

Another object of present invention is to use the MBL composition to reduce the likelihood of hyperbillirubinemia in a critically ill patient.

Another object of present invention is to use the MBL composition to reduce the liklihood for blood stream infections in a critically ill patient.

Another object of present invention is to use the MBL composition to reduce the likelihood of disturbance in markers of inflammations and/or inflammatory response in a critically ill patient.

Another object of present invention is to use the MBL composition to reduce the use of antibiotics in a critically ill patient.

Another object of present invention is to use the MBL composition to reduce the likelihood of a critically ill patient having repetitive positive EMGs.

Another object of present invention is to use the MBL composition to reduce the amount of red cell transfusion in a critically ill patient.

Another object of present invention is to use the MBL composition to reduce the need for invasive treatment in a critically ill patient.

Compositions

The medicament containing MBL may be produced by using the eluant obtained from the affinity chromatography as such It is however preferred that the eluant is subjected to further purification steps before being used.

In addition to the MBL polypeptide oligomers, the medicament may comprise a pharmaceutically acceptable carrier substance and/or vehicles. In particular, a stabilising agent may be added to stabilise the MBL proteins. The stabilising agent may be a sugar alcohol, saccharides, proteins and/or aminoacids. An example of a stabilising agent may be albumin.

Other conventional additives may be added to the medicament depending on administration form for example. In one embodiment the medicament is in a form suitable for injections. Conventional carrier substances, such as isotonic saline, may be used.

In another embodiment the medicament is in a form suitable for pulmnonal administration, such as in the form of a powder for inhalation or creme or fluid for topical application.

The route of administration may be any suitable route, such as intravenously, intramuscularly, subcutanously or intradermally. Also, pulmonal or topical administration is envisaged by the present invention.

In particular the MBL composition may be administered to prevent and/or treat “ICU complications” in patients having clinical symptoms associated with congenital or acquired MBL deficiency or being at risk of developing such symptoms.

The MBL composition may also be administered simultaneously, sequentially or separately with another treatment.

The MBL composition is administered in suitable dosages, in particularly it is administered at ICU admission and maintained once, twice or thrice a week at least during a part of the stay at ICU, preferably during the whole ICU period.

Normally from 1-100 mg is administered per dosage, such as from 2-10 mg, mostly from 5-10 mg per dosage. For other indications the dosage regime may vary.

The use of a MBL composition may also be in a kit-of-parts further comprising an anti-fungal, anti-yeast, anti-bacterial and/or anti-viral medicament. The anti-viral medicament may be a medicament capable of virus attenuation and/or elimination.

The invention also relates to an aspect of using a measurement of the MBL level as a prognostic marker for the risk of the individual of acquiring ICU complications and thereby an indicative of the need for treatment. In particular a MBL level below 500 ng/ml is a prognostic marker indicative for treatment with MBL.

Thus, the present invention also relates to a method of using a MBL polypeptide composition for preventing and/or reducing “ICU complications” in an individual, the method comprising the steps of:
i) determining serum levels of MBL polypeptide in an individual,
ii) estimating the probability of the occurrence of ICU complications in the individual, and optionally, administering a MBL polypeptide composition to the individual.

The MBL level is measured in serum or plasma, and may be determined by time resolved immunofluorescent assay (TRIFMA), ELISA, RIA or nephelometry.

Also the MBL levels may be inferred from analysis of genotypes of the MBL genes.

The invention has now been explained and accounted for in various aspects, but additionally it will be illustrated below by FIGS. 1 and 2 and table 1 and the non-limiting examples of preferred embodiments.

### FIGURE LEGENDS

FIG. 1:

Serial measurements of mannan-binding lectin (MBL) concentrations in patients receiving prolonged (>5 days) intensive care. Bars represents medians, boxes IQRs and whiskers the 10^{th} and 90^{th} percentiles. P value refers to Friedman's test for several related samples

FIG. 2:

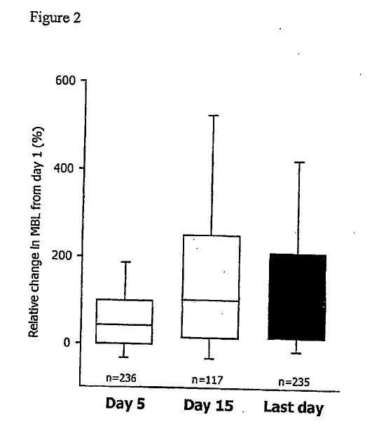

Relative change in MBL concentrations from day 1 (Δ%) in patients receiving prolonged intensive care.

### EXAMPLE

The following example demonstrates the results of an examination of the influence of MBL deficiency on outcome for individuals admitted to intensive care units.

Study Population

The study encompasses examination of 243 patients on mechanical ventilation admitted to the Department of Intensive Care Medicine, University of Leuven, Leuven, Belgium. All patients included in this study received intensive care for more than 5 days.

Blood stream infection was defined by a blinded investigator as the presence of bacterial pathogens, excluding contamination according to Strict criteria (Weinstein M P et al Clin. Infect. Dis. 1997; 24 (4): 584-602.), in blood cultures obtained when central body temperature steeply rose above 38.5° C. The use of antibiotics was recorded as the total number of days on any systemic antibiotic treatment. The number of days during which leukopenia (≦4000 cells/μl) or leukocytosis (≧12000 cells/μl) was present, and the number of days during which an episode of hypothermia (≦36° C.) or hyperthermia (≧38° C.) occurred, were also analysed. The incidence of acute renal failure requiring renal replacement therapy was recorded. Weekly EMG screenings were performed for the diagnosis of critical illness polyneuropathy. The cause of death for all patients who died was established clinically by the attending physician and confirmed on post-mortem examination by a pathologist who was unaware of treatment assignment.

MBL Measurements

Blood samples were drawn within 24 h after admission to the ICU (baseline), and subsequently on days 5 and 15 and/or the last day of intensive care (ie. the day of discharge or death) for determination of serum MBL.

Serum MBL concentrations were measured using time-resolved immunofluorometric assay (IRMA) (Thiel S et al, Immunology 2002; 204). Microtiter wells (fluoroNunc, Nunc, Denmark) were coated with mannan followed by incubation with diluted test samples and standards.

After washing, europium labelled monoclonal anti-MBL antibody (131-1, Immuno-lex, Denmark, labelled with europium using reagents from Wallac Oy, Finland) was added, and after incubation and washing fluorescence enhancement solution was added (Wallac) and the plates were read on a time resolved fluorometre (Delfia 1232, Wallac). The calibration curve was made using dilutions of one plasma, which was kept alliquoted at −80° C. The concentration of MBL in this plasma (3.6 μg/ml) was determined by comparison with highly purified MBL, which was quantified by quantitative amino acid analysis.

An alternative TRIFMA for analysing the MBL is coating Microtitre wells (fluoro-Nunc, Nunc, Karnstrup, Denmark) with antibody by incubation overnight at room temperature with 500 ng anti-humanMBL antibody (Mab 131-1, Statens Serum Institut, Copenhagen, Denmark) in I 00 pfPBS (0.14 M NaCl, 10 mM phosphate, pH 7.4). After wash with Tween-containing buffer (TBS, 0.14 M NaCl, 10 mM Tris/HCI, 7.5 mM NaN3, pH 7.4 with 0.05% Tween20) test samples (plasma 1/20) and calibrator dilutions can be added in TBS/Tween with extra NaCl to 0.5 M and 10 mM EDTA.

After overnight incubation at 40C and wash, the developing europium-labelled anti-body (12.5 ng Mab 131-1 labelled with the Eu-containing chelate, isothiocyanato-benzoyl-diethylene-trianine-tetra acetic acid, according to the manufacturer, Wallac,Turku, Finland) can be added in TBS/Tween with 25 pM EDTA.

Following incubation for 2 h and wash, fluorescence enhancement solution is added (Wallac) and the plates are read on a time resolved fluorometre (Delfia 1232, Wal-lac). The calibration curve is made using dilutions of one plasma, which is kept al-liquoted at −80 C.

Collected blood for TRIFMA analysis is drawn into evacuated glass tubes containing EDTA (final concentration about 10 mM). The plasma is aliquoted and kept at −80° C. until assay. Plasma samples are similarly obtained from healthy blood donors. The patient are free of infections at the time of blood sampling.

Statistical Analysis

Changes in MBL concentrations over time during the ICU-stay were analysed by Friedman's test. Spearmian correlation with two-tailed probability values was used to estimate the strength of association between variables. The impact of baseline MBL level on outcome variables (mortality, acute renal failure, bacteremia, prolonged need for antibiotic treatment, polyneuropathy) was assessed by multivariate logistic regression analysis. In addition, multivariate logistic regression analysis was used to assess whether the changes in MBL over time explained clinical outcome variables. Data are given as medians with interquartile ranges unless specified otherwise and statistical significance was assumed for P<0.05. All statistical calculations were performed with Statview 5.0.1. for Macintosh (SAS Institute Inc., North Carolina, USA).

Results

Clinical Patient Characteristics and Outcome

Of the 243 patients included in the study 49 died during intensive care. The cause of death was multiple-organ failure with or without a proven septic focus, acute cardiovascular collapse and severe brain damage.

Bacteraemia occurred in 25% of the patients. The patients received treatment with antibiotics for a median duration of 12 (IQR 6-21) days, leucopenia or leucocytosis was present for a median of 6 (IQR 2-13) days and hypo- or hyperthermia for a median 10 (IQR 5-16) days.

Serum MBL Concentrations

Upon ICU admission, the average serum MBL concentration was 820 [IQR 241-1518] μg/l which is comparable with the level documented in healthy Danish and British subjects. The number of patients with a baseline MBL level below 500 ng/ml and below 50 ng/ml was 40% and 8.9%, respectively.

Analysis of the treated patients, non-survivors revealed significantly lower baseline MBL concentrations as compared with survivors (387 [IQR 190-1289]) μg/l vs. 897 [IQR 246-1686] μg/l, respectively; P=0.04, Table 1). The fraction of patients with MBL concentrations below 500 ng/ml was 54% among non-survivors as compared to 36% among survivors (P=0.02). The fraction of patients with MBL concentrations below 250 ng/ml was 34% among non-survivors as compared to 25% among survivors, and the fraction of patients with MBL concentrations below 50 ng/ml was 14% among non-survivors as compared to 7% among survivors.

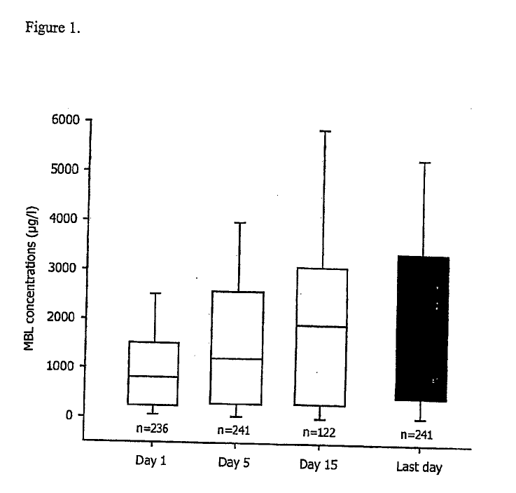

The MBL concentrations increased significantly with time in intensive care (P<0.0001, FIG. 1). This rise was independent of the baseline MBL concentration and mostly attributable to the survivors. Patients who developed bacteremia revealed a lower relative increase in MBL levels on day 15 compared to those who did not develop bacteremia (P≦0.02).

Discussion

Protracted critical illness is associated with substantial metabolic and immunological derangement and a high risk of death (Van den Berghe G. et al 2000; 143 (1): 1-13; Van den Berghe G, et al N Engl J Med 2001;345(19):1359-67; Takala J, et al. N Engl J Med 1999;341(11):785-92) ). We observed that low on-admission concentrations of MBL may predict a poor outcome among patients treated with intensive care.

More than two-thirds of patients admitted to intensive care units develop signs of the systemic inflammatory response syndrome (SIRS) (Brun-Buisson C. Intensive Care Med 2000; 26: S64-S74 ), either caused by infection or tissue damage, and a substantial number of these patients progress to shock and multiple-organ failure.

A number of publications have reported a possible association between low levels of MBL and increased risk of infections particularly in patients who are immunocompromised, such as children with immature antibody repertoire (Koch,-A et al. JAMA Mar. 14, 2001; 285(10): 1316-21), patients with AIDS (Kelly,-P. et al Gastroenterology. November 2000; 119(5): 1236-42.Gastroenterology), or patients with malignancies receiving chemotherapy or stem cell transplantation (Neth,-O; et al Lancet. Aug. 25, 2001; 358 (9282): 614-8; Peterslund,-N-A; Koch,-C; Jensenius,-J-C; Thiel,-S Lancet. Aug. 25, 2001 ; 358(9282): 637-8; Mullighan,-Charles-G; et al. Blood May 15, 2002; 99(10): 3524-9.).

In present invention the current analysis of the 243 ICU treated patients that were not previously immunocompremised revealed that MBL levels on admission were almost three times higher in survivors than in non-survivors, in favour of a vulnerability associated with low MBL levels in critically ill patients who are immunocompetent. The association between low levels of MBL and outcome of treated ICU patients was not restricted to severe MBL deficiency but was evident even when using a concentration of <500 ng/ml as a cut-off level for functional MBL deficiency as previously suggested by Peterslund et al (Lancet. Aug. 25, 2001; 358(9282): 637-8).

In conclusion low levels of MBL may predict a poor outcome in protracted critical illness.

TABLE 1
Serial measurements of MBL concentrations in patients
undergoing prolonged intensive care treatment.

| | | Died in ICU | ICU survivor | |
|---|---|---|---|---|
| All patients | | n = 49 | n = 194 | P |
| MBL concentrations | Day 1 | 387 (190-1287) | 897 (246-1686) | 0.045 |
| (μg/l, median | Day 5 | 460 (158-2140) | 1321 (346-2706) | 0.012 |
| [IQR]) | Day 15 | 1855 (181-2594) | 1934 (322-3574) | 0.376 |
| | Last Day | 990 (240-2408) | 1960 (569-3848) | 0.002 |

1-50. (canceled)

51. A method comprising a use of mannan-binding lectin (MBL) for the treatment of a patient suffering from systemic inflammatory response syndrome (SIRS) and/or a patient in need of transplantation and/or for reducing the risk in a patient of acquiring SIRS.

52. The method according to claim 51, wherein the patient is in need of a transplantation.

53. The method according to claim 51, to prevent the patient from acquiring SIRS.

54. The method according to claim 51, wherein the patient is critically ill, wherein a critically ill patient is a patient which has sustained or are at risk of sustaining acutely life-threatening single or multiple organ system failure due to disease or injury, a patient who is being operated and where complications supervene, and a patient who has been operated in a vital organ within the last week or subject to major surgery within the last week.

55. The method according to claim 51, wherein said MBL treatment is administered to increase the survival rate in the intensive care unit (ICU).

56. The method according to claim 51, wherein said MBL treatment is administered to reduce the time that a patient stays within the hospital, for example within the intense care unit.

57. The method according to claim 54, wherein the patient suffers from post-surgical critical illness.

58. The method according to claim 54, wherein the patient suffers from post-traumatic critical illness.

59. The method according to claim 51, wherein the MBL polypeptide monomer is a mammalian polypeptide monomer.

60. The method according to claim 59, wherein said mammalian MBL polypeptide monomer is a human polypeptide monomer.

61. The method according to claim 51, wherein said MBL comprises oligomers, wherein said oligomers are preferably selected from the group of oligomers consisting of trimers, tetramers, pentamers and/or hexamers.

62. The method according to claim 51, wherein the MBL comprises at least one mannan-binding lectin (MBL) polypeptide monomer, or at least one mannan-binding lectin (MBL) polypeptide oligomer comprising or at least one mannan-binding lectin (MBL) polypeptide monomer.

63. The method according to claim 51, wherein MBL comprises at least one mannan-binding lectin (MBL) polypeptide oligomer comprising at least one mannan-binding lectin (MBL) polypeptide monomer.

64. The method according to claim 51, wherein the medicament is used in such way that the blood MBL level in the patient is kept above 250 ng/ml.

65. The method according to claim 51, wherein the medicament is used in such way that the blood MBL level in the patient is kept above 500 ng/ml.

66. The method according to claim 51, wherein the medicament is used in such way that the blood MBL level in the patient is kept above 1000 ng/ml.

67. The method according to claim 51, wherein the medicament is used in such way that the blood MBL level in the patient is kept between 1000 ng/ml and 2000 ng/ml.

68. The method according to claim 51, wherein the medicament is administered to the individual prior to another treatment at ICUs.

69. The method according to claim 51, wherein the medicament is administered to the individual simultaneously, sequentially or separately with another treatment.

70. The method according to claim 51, wherein the medicament is administered to the individual prior to during or after said other treatment.

71. The method according to claim 51, wherein the patient has MBL polypeptide serum levels below a predetermined minimum MBL polypeptide serum level, wherein the predetermined level is a concentration of 500 ng/ml.

72. The method according to claim 51, wherein said treatment is to reduce the risk of a fatal outcome during intensive care treatment of an individual.
